(19) United States
(12) Patent Application Publication
(10) Pub. No.: US 2003/0135936 A1
(43) Pub. Date: Jul. 24, 2003

(54) Agent for dyeing or coloring and simultaneously protecting hair

(76) Inventors: Astrid Kleen, Erkrath (DE); Frank Naumann, Duesseldorf (DE); Horst Hoeffkes, Duesseldorf (DE)

Correspondence Address: Henkel Corporation, Law Department, Suite 200, 2500 Renaissance Blvd., Gulph Mills, PA, 19406, US

(21) Appl. No.: 10/319,905

(22) Filed: Dec. 13, 2002

Related U.S. Application Data
(63) Continuation of application No. PCT/EP01/06560, filed on Jun. 9, 2001.

(30) Foreign Application Priority Data
Jun. 20, 2000 (DE) 100 30 313.7
Apr. 26, 2001 (DE) 101 20 304.7

(51) Int. Cl. A61K 7/13

(52) U.S. Cl. 8/405; 8/409; 8/424

(57) ABSTRACT

There are provided novel preparations for tinting or dyeing hair which contain vitamin B6 derivatives.

### CROSS REFERENCE TO RELATED APPLICATIONS

This application is a continuation under 35 U.S.C. §365 (c) and §120 of International Application No. PCT/EP01/06560 filed Jun. 9, 2001 and under §119 of German Application Nos. 100 30 313.7 filed Jun. 20, 2000 and 101 20 304.7 filed Apr. 26, 2001.

### SUMMARY OF THE INVENTION

This invention relates to a preparation and a process for coloring or tinting and caring for keratin fibers, more particularly human hair, and to the use of vitamin B6 derivatives in a corresponding process.

### BACKGROUND OF THE INVENTION

Preparations for tinting and coloring hair are an important type of cosmetic product. They may be used to lighten or darken the natural color of hair according to the wishes of the particular user, to obtain a completely different color or to cover unwanted color tones, for example grays. Conventional hair colorants are formulated either on the basis of oxidation dyes or on the basis of substantive dyes. In many cases, combinations of oxidation dyes and substantive dyes are also used to obtain special shades.

Good dyes are distinguished by high coloring strength. In addition, they are expected to be resistant to perspiration, heat, permanent waving, washing and light and to be toxicologically and dermatologically safe. It is also of advantage if the substances show high solubility in various basic formulations.

Colorants based on oxidation dyes lead to brilliant and permanent colors. However, they do involve the use of strong oxidizing agents such as, for example, hydrogen peroxide solutions. This can damage the hair to be colored. Skin contact with these colorants can lead to unwanted reactions in very sensitive people.

Colorants based on substantive dyes do not require oxidizing agents and can be even be formulated at pH values around the neutral point.

Besides coloring or tinting, hair care is an important aspect of hair cosmetology. Thus, the application of hair-care formulations containing keratin-structuring ingredients can counteract the damaging effects produced, for example, by alkaline and/or keratin-reducing preparations.

Pyridoxine and other compounds belonging to the vitamin B6 group have already been proposed for use as components in hair tonics for reducing refatting and for stimulating hair growth. EP 0678293 A2 proposes topical compositions containing pyridoxine tripropionate for treating the hair and skin. EP 001079 A1 describes describes anti-seborrheic cosmetic compositions containing pyridoxine tripalmitate as their active ingredient.

Hair colorants containing derivatives of pyridoxine, pyridoxal or pyridoxamine as a keratin-restructuring agent have not hitherto been known to the expert.

It has now surprisingly been found that the use of derivatives of pyridoxine, pyridoxal and pyridoxamine in a colorant containing at least one substantive dye has a restructuring effect on hair keratin. This effect is even developed during coloring.

### DETAILED DESCRIPTION OF THE INVENTION

In a first embodiment, therefore, the present invention relates to a preparation for coloring keratin fibers, more particularly human hair, containing at least one substantive dye, characterized in that the colorant additionally contains at least one compound corresponding to formula (I):

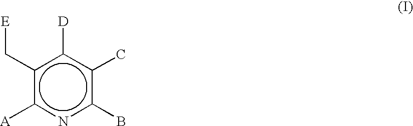

in which

A and B independently of one another represent hydrogen, halogen, a C₁₋₄ alkyl group, a C₃₋₆ cycloalkyl group, a C₁₋₄ monohydroxyalkyl group, a C₂₋₄ oligohydroxyalkyl group, a C₁₋₄ aminoalkyl group, a group —OR or a group —NR¹R², where R¹ and R² independently of one another represent hydrogen, a C₁₋₄ alkyl group or a C₁₋₄ monohydroxyalkyl group or R¹ and R² together with the nitrogen atom form a saturated ring,

C represents a group —OR, —NR¹R², —OP(O)(OR³)₂, a C₁₋₄ monohydroxyalkyl group, a C₂₋₄ oligohydroxyalkyl group or a C₁₋₄ alkyl group,

D represents a group —OR, a carboxy group, a C₁₋₂₂ alkoxycarbonyl group, a formyl group, a group —CH₂OR or a group —CH₂—NR²,

E represents a group —OR, —OP(O)(OR³)₂, a C₁₋₄ monohydroxyalkyl group or a C₂₋₄ oligohydroxyalkyl group,

R representing hydrogen, a C₁₋₄ alkyl group, a C₁₋₂₂ acyl group, a hydroxy-C₂₋₂₂-acyl group, a C₂₋₁₀ carboxyacyl group, a C₃₋₁₀ oligocarboxyacyl group, an oligocarboxymonohydroxy-C₃₋₁₀-acyl group, an oligocarboxyoligohydroxy-C₃₋₁₀-acyl group, a C₃₋₈ cycloalkyl group, a C₁₋₄ monohydroxyalkyl group, a C₂₋₄ oligohydroxyalkyl group, an aryl group which may contain a hydroxy, nitro or amino group, a heteroaromatic group or a group —CH₂CH₂NR¹R², where R¹ and R² are as defined above,

R³ representing hydrogen or a C₁₋₅ alkyl group,

or one of the corresponding physiologically compatible salts.

Compounds corresponding to formula (I) in which one of the two groups A and B is hydrogen are preferred. Particularly preferred compounds corresponding to formula (I) are those in which one of the two groups A and B is hydrogen and the other group is a C₁₋₄ alkyl group.

Other preferred compounds of formula (I) are those in which C is a hydroxy group, a C₁₋₄ monohydroxyalkyl group or a C₂₋₄ oligohydroxyalkyl group.

Preferred compounds of formula (I) are those in which D is a hydroxymethyl group, a hydroxy group, a carboxy group, a group —CH₂—NR₂ or a formyl group.

Other preferred compounds of formula (I) are those in which E is a hydroxy group, a C₁₋₄ monohydroxyalkyl group or a group —OP(O)(OH)₂.

Particularly preferred compounds corresponding to formula I are pyridoxine (A=H, B=CH₃, C=OH, D=CH₂OH, E=OH), pyridoxal (A=H, B=CH₃, C=OH, D=CHO, E=OH), pyridoxal-5′-phosphate (A=H, B=CH₃, C=OH, D=CHO, E=OP(O)(OH)₂) and pyridoxamine (A=H, B=CH₃, C=OH, D=CH₂NH₂, E=OH).

Examples of C₁₋₄ alkyl groups in the compounds according to the invention are methyl, ethyl, n-propyl, isopropyl, n-butyl and tert.butyl. Preferred alkyl groups are methyl and ethyl. Methyl is a particularly preferred alkyl group. Preferred C₃₋₆ cycloalkyl groups are cyclopropyl, cyclobutyl, cyclopentyl, cyclohexyl. Cyclohexyl and cyclopentyl are particularly preferred groups. Preferred C₁₋₄ monohydroxyalkyl groups are the groups hydroxymethyl, 2-hydroxyethyl, 3-hydroxypropyl or 4-hydroxybutyl; hydroxymethyl and 2-hydroxyethyl are particularly preferred monohydroxyalkyl groups. A preferred C₂₋₄ oligohydroxyalkyl group is the 1,2-dihydroxyethyl group. Preferred C₁₋₂₂ acyl groups are, for example, acetyl, propionyl, butyryl, valeryl, capryl, lauryl, myristyl, palmityl, stearyl, linolyl, behenyl. Examples of a hydroxy-C₂₋₂₂-acyl group are salicylic acid or lactic acid. Preferred C₂₋₁₀ carboxyacyl groups are derived, for example, from malonic acid, succinic acid or adipic acid. One example of a preferred C₃₋₁₀ oligocarboxyacyl group is glyceric acid. A preferred oligocarboxymonohydroxy-C₃₋₁₀-acyl group is derived, for example, from citric acid or malic acid. Preferred oligocarboxyoligohydroxy-C₃₋₁₀-acyl groups are derived, for example, from tartaric acid. According to the invention, preferred halogen substituents are fluorine, chlorine, bromine and iodine; chlorine and bromine are particularly preferred. Physiologically compatible salts in the context of the invention are salts of inorganic or organic acids, for example hydrochlorides, sulfates or hydrobromides. According to the invention, the other terms used are derived from the definitions given here.

The ester derivatives of the compounds corresponding to formula (I) also have physiological and hair-structure-improving properties. This applies in particular to the esters of pyridoxine which can be converted by hydrolysis into pyridoxine. In addition, the ester derivatives acquire improved lipid solubility compared with the non-esterified derivatives. Other examples of carboxylic acid ester derivatives of pyridoxine are derived from the carboxylic acids, such as formic acid, acetic acid, propionic acid, butyric acid, isobutyric acid, valeric acid, isovaleric acid, pivalic acid, oxalic acid, malonic acid, succinic acid, glutaric acid, glyceric acid, glyoxylic acid, adipic acid, pimelic acid, suberic acid, azelaic acid, sebacic acid, propiolic acid, crotonic acid, isocrotonic acid, elaidic acid, maleic acid, fumaric acid, muconic acid, citraconic acid, mesaconic acid, camphor acid, benzoic acid, o,m,p-phthalic acid, naphthoic acid, toluylic acid, hydratropic acid, atropic acid, cinnamic acid, isonicotinic acid, nicotinic acid, bicarbamic acid, 4,4′-dicyano-6,6′-binicotinic acid, 8-carbamoyloctanoic acid, 1,2,4-pentanetricarboxylic acid, 2-pyrrole carboxylic acid, 1,2,4,6,7-naphthalene pentaacetic acid, malonaldehydic acid, 4-hydroxyphthalamidic acid, 1-pyrazole carboxylic acid, gallic acid or propane tricarboxylic acid, and from dicarboxylic acids selected from the group consisting of compounds corresponding to general formula (II):

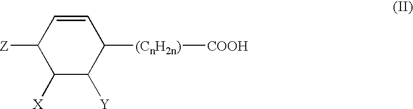

in which Z represents a linear or branched alkyl or alkenyl group containing 4 to 12 carbon atoms, n is a number of 4 to 12 and one of the two groups X and Y represents a COOH group and the other represents hydrogen or a methyl or ethyl group, dicarboxylic acids corresponding to general formula (II) which additionally contain 1 to 3 methyl or ethyl substituents on the cyclohexene ring and dicarboxylic acids which formally are formed from the dicarboxylic acids (II) by addition of one molecule of water onto the double bond in the cyclohexene ring.

The preparations according to the invention preferably contain the compound corresponding to formula (I) in quantities of 0.05 to 5.0% by weight, based on the colorant. Quantities of 0.1 to 2% by weight are particularly preferred.

In a first preferred embodiment of the invention, the preparations are merely intended to produce temporary coloring of the fibers. Corresponding preparations are often referred to as tinting preparations. This embodiment also encompasses, for example, hair treatment preparations with which the hair is not only to be temporarily colored, but also shaped into a certain style. Such preparations are known as tinting gels or foams.

Since the coloring of the keratin fibers with tinting preparations normally takes place without the assistance of oxidizing components, more particularly hydrogen peroxide, the preparations according to the invention of this embodiment are preferably free from oxidation dye precursors.

The preparations according to the invention may be formulated as coloring creams, tinctures, lotions, foams or aerosols.

The invention encompasses preparations which also contain a substantive dye in addition to the compound corresponding to formula (I). In a preferred embodiment, the preparations according to the invention contain several, more particularly 2 to 10, substantive dyes.

Substantive dyes are typically nitrophenylenediamines, nitroaminophenols, azo dyes, anthraquinones or indophenols. Preferred substantive dyes are the compounds known under the International names or commercial names of HC Yellow 2, HC Yellow 4, HC Yellow 5, HC Yellow 6, HC Yellow 12, HC Orange 1, Disperse Orange 3, HC Red 1, HC Red 3, HC Red 10, HC Red 11, HC Red 13, HC Red BN, HC Blue 2, HC Blue 12, Disperse Blue 3, HC Violet 1, Disperse Violet 1, Disperse Violet 4, Acid Violet 43, Disperse Black 9 and Acid Black 52 and also 1,4-diamino-2-nitrobenzene, 2-amino-4-nitrophenol, 1,4-bis-(β-hydroxyethyl)-amino-2-nitrobenzene, 3-nitro-4-(β-hydroxyethyl)-aminophenol, 2-(2′-hydroxyethyl)-amino-4,6-dinitrophenol, 1-(2′-hydroxyethyl)-amino-4-methyl-2-nitro-benzene, 1-amino-4-(2′-hydroxyethyl)-amino-5-chloro-2-nitrobenzene, 4-amino-3-nitrophenol, 1-(2′-ureidoethyl)-amino-4-nitrobenzene, 4-amino-2-nitrodiphenylamine-2′-carboxylic acid, 6-nitro-1,2,3,4-tetrahydroquinoxaline, 2-hydroxy-1,4-naphthoquinone, picramic acid and salts thereof, 2-amino-6-chloro-4-nitrophenol, 4-ethylamino-3-nitrobenzoic acid and 2-chloro-6-ethylamino-1-hydroxy-4-nitrobenzene.

According to the invention, cationic substantive dyes may also be used as substantive dyes. Particularly preferred are

(a) cationic triphenylmethane dyes such as, for example, Basic Blue 7, Basic Blue 26, Basic Violet 2 and Basic Violet 14,

(b) aromatic systems substituted by a quaternary nitrogen group such as, for example, Basic Yellow 57, Basic Red 76, Basic Blue 99, Basic Brown 16 and Basic Brown 17 and

(c) substantive dyes containing a heterocycle with at least one quaternary nitrogen atom as disclosed, for example, in EP-A2 998 908 in claims 6 to 11 and in WO 95/01772 and WO 95/15144. Reference is specifically made to the disclosures of these documents.

Preferred cationic substantive dyes of group (c) are, in particular, the following compounds:

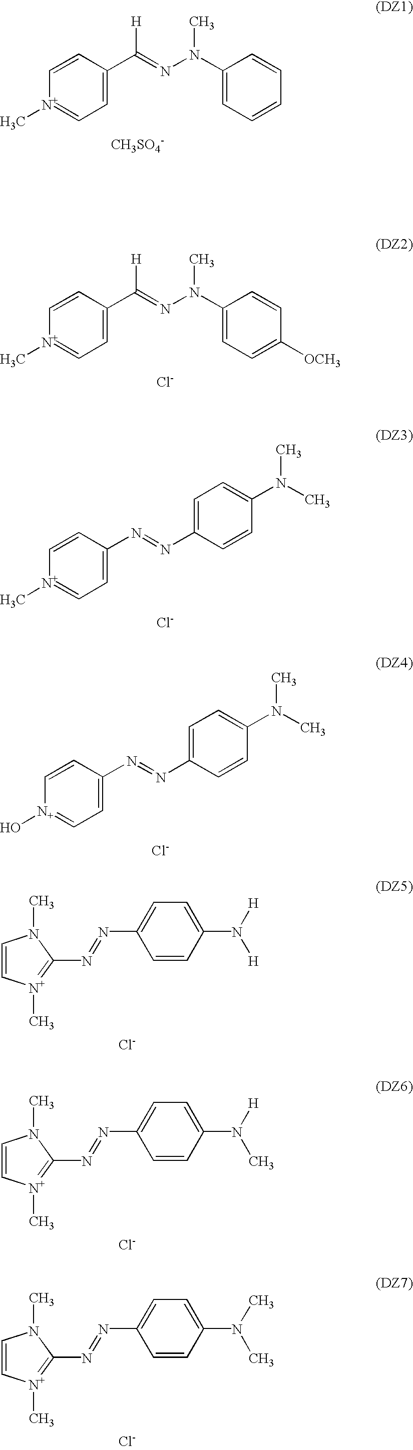

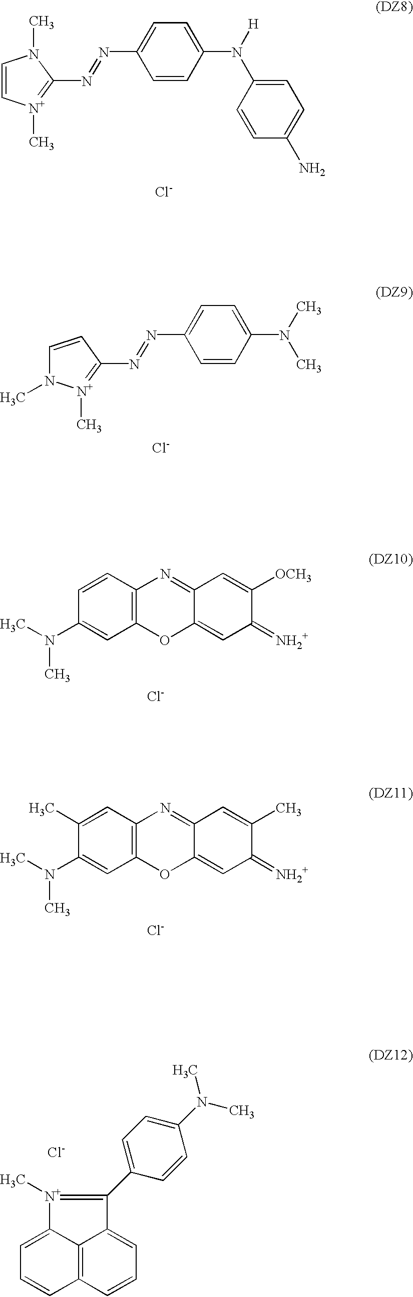

The compounds corresponding to formula (DZ1), (DZ5) and (DZ7) are most particularly preferred cationic substantive dyes of group (c). According to the invention, the cationic substantive dyes marketed under the name of Arianor® are particularly preferred substantive dyes.

The preparations according to the invention of this embodiment preferably contain the substantive dyes in a quantity of 0.01 to 20% by weight, based on the colorant as a whole.

The preparations according to the invention may also contain naturally occurring dyes such as, for example, henna red, henna neutral, henna black, chamomile blossom, sandalwood, black tea, black alder bark, sage, logwood, madder root, catechu, sedre and alkanet.

In a second preferred embodiment, the preparations according to the invention additionally contain at least one oxidation dye precursor of the primary intermediate type. These generally colorless compounds react under the influence of oxidizing agents or atmospheric oxygen, optionally in the presence of special enzymes or metal ions as catalyst, to form the required dyes. However, combinations of several primary intermediates are generally used, above all to form natural hair colors. In addition, so-called secondary intermediates are generally used and react with the primary intermediates under the influence of oxidizing agents to form new colors or to shade the color. According to the invention, one or more secondary intermediates may be used in combination with one or more primary intermediates.

According to the invention, preferred primary intermediates are p-phenylenediamine, p-toluylenediamine, p-aminophenol, o-aminophenol, 1-(2′-hydroxyethyl)-2,5-diaminobenzene, N,N-bis-(2-hydroxyethyl)-p-phenylenediamine, 2-(2,5-diaminophenoxy)-ethanol, 1-phenyl-3-carboxyamido-4-amino-5-pyrazolone, 4-amino-3-methylphenol, 2,4,5,6-tetraaminopyrimidine, 2-hydroxy-4,5,6-triaminopyrimidine, 4-hydroxy-2,5,6-triaminopyrimidine, 2,4-dihydroxy-5,6-diaminopyrimidine, 2-dimethylamino-4,5,6-triaminopyrimidine, 2-hydroxymethylaminomethyl-4-aminophenol, bis-(4-aminophenyl)-amine, 4-amino-3-fluorophenol, 2-aminomethyl-4-aminophenol, 2-hydroxymethyl-4-aminophenol, 4-amino-2-((diethylamino)-methyl)-phenol, bis-(2-hydroxy-5-aminophenyl)-methane, N,N′-bis-(4′-aminophenyl)-1,4-diazacycloheptane, 1,3-bis-(N-(2-hydroxyethyl)-N-(4-aminophenylamino))-2-propanol, 4-amino-2-(2-hydroxyethoxy)-phenol, 1,10-bis-(2,5-diaminophenyl)-1,4,7,10-tetraoxadecane and 4,5-diamino-pyrazole derivatives according to EP 0 740 931 or WO 94/08970, for example 4,5-diamino-1-(2′-hydroxyethyl)-pyrazole.

According to the invention, preferred secondary intermediates are

m-aminophenol and derivatives thereof such as, for example, 5-amino-2-methylphenol, N-cyclopentyl-3-aminopenol, 3-amino-2-chloro-6-methylphenol, 2-hydroxy-4-aminophenoxyethanol, 2,6-dimethyl-3-aminophenol, 3-trifluoroacetylamino-2-chloro-6-methylphenol, 5-amino-4-chloro-2-methylphenol, 5-amino-4methoxy-2-methylphenol, 5-(2′-hydroxyethyl)-amino-2-methylphenol, 3-(diethylamino)-phenol, N-cyclopentyl-3-aminophenol, 1,3-dihydroxy-5-(methylamino)-benzene, 3-(ethylamino)-4-methylphenol and 2,4-dichloro-3-aminophenol,

o-aminophenol and derivatives thereof,

m-diaminobenzene and derivatives thereof such as, for example, 2,4-diaminophenoxyethanol, 1,3-bis-(2′,4′-diaminophenoxy)-propane, 1-methoxy-2-amino-4-(2′-hydroxyethylamino)-benzene, 1,3-bis-(2′,4′-diaminophenyl)-propane, 2,6-bis-(2′-hydroxyethylamino)-1-methylbenzene and 1-amino-3-bis-(2′-hydroxyethyl)-aminobenzene,

o-diaminobenzene and derivatives thereof such as, for example, 3,4-diaminobenzoic acid and 2,3-diamino-1-methylbenzene,

di- and trihydroxybenzene derivatives such as, for example, resorcinol, resorcinol monomethyl ether, 2-methyl resorcinol, 5-methyl resorcinol, 2,5-dimethyl resorcinol, 2-chlororesorcinol, 4-chlororesorcinol, pyrogallol and 1,2,4-trihydroxybenzene,

pyridine derivatives such as, for example, 2,6-dihydroxypyridine, 2-amino-3-hydroxypyridine, 2-amino-5-chloro-3-hydroxypyridine, 3-amino-2-methylamino-6-methoxypyridine, 2,6-dihydroxy-3,4-dimethylpyridine, 2,6-dihydroxy-4-methylpyridine, 2,6-diaminopyridine, 2,3-diamino-6-methoxypyridine and 3,5-diamino-2,6-dimethoxypyridine,

naphthalene derivatives such as, for example, 1-naphthol, 2-methyl-1-naphthol, 2-hydroxymethyl-1-naphthol, 2-hydroxyethyl-1-naphthol, 1,5-dihydroxynaphthalene, 1,6-dihdroxynaphthalene, 1,7-dihdroxynaphthalene, 1,8-dihdroxynaphthalene, 2,7-dihdroxynaphthalene and 2,3-dihdroxynaphthalene,

morpholine derivatives such as, for example, 6-hydroxybenzomorpholine and 6-aminobenzomorpholine,

quinoxaline derivatives such as, for example, 6-methyl-1,2,3,4-tetrahydroquinoxaline,

pyrazole derivatives such as, for example, 1-phenyl-3-methylpyrazol-5-one,

indole derivatives such as, for example, 4-hydroxyindole, 6-hydroxyindole and 7-hydroxyindole,

pyrimidine derivatives such as, for example, 4,6-diaminopyrimidine, 4-amino-2,6-dihydroxypyrimidine, 2,4-diamino-6-hydroxypyrimidine, 2,4,6-trihydroxypyrimidine, 2-amino-4-methylpyrimidine, 2-amino-4-hydroxy-6-methylpyrimidine and 4,6-dihydroxy-2-methylpyrimidine or

methylenedioxybenzene derivatives such as, for example, 1-hydroxy-3,4-methylenedioxybenzene, 1-amino-3,4-methylenedioxybenzene and 1-(2′-hydroxyethyl)-amino-3,4-methylenedioxybenzene.

Particularly preferred secondary intermediates are 1-naphthol, 1,5-, 2,7- and 1,7-dihydroxynaphthalene, 3-aminophenol, 5-amino-2-methylphenol, 2-amino-3-hydroxypyridine, resorcinol, 4-chlororesorcinol, 2-chloro-6-methyl-3-aminophenol, 2-methyl resorcinol, 5-methyl resorcinol, 2,5-dimethyl resorcinol and 2,6-dihydroxy-3,4-dimethylpyridine.

Besides the compounds corresponding to formula (I), the primary intermediates and optionally the secondary intermediates, the preparations according to the invention of this embodiment preferably contain several substantive dyes. Reference is made at this juncture to the foregoing observations.

The colorants according to the invention may additionally contain precursors of natural dyes, more particularly indoles and indolines, and physiologically compatible salts thereof. Preferred examples are 5,6-dihydroxyindole, N-methyl-5,6-dihydroxyindole, N-ethyl-5,6-dihydroxyindole, N-propyl-5,6-dihydroxyindole, N-butyl-5,6-dihydroxyindole, 5,6-dihydroxyindole-2-carboxylic acid, 6-hydroxyindole-6-aminoindole and 4aminoindole. Also preferred are 5,6-dihydroxyindoline, N-methyl-5,6-dihydroxyindoline, N-ethyl-5,6-dihydroxyindoline, N-propyl-5,6-dihydroxyindoline, N-butyl-5,6-dihydroxyindoline, 5,6-dihydroxyindoline-2-carboxylic acid, 6-hydroxyindoline, 6-aminoindoline and 4-aminoindoline.

Neither the oxidation dye precursors nor the substantive dyes nor the precursors of natural dyes have to be single compounds. On the contrary, other components may be present in small quantities in the hair colorants according to the invention due to the processes used to produce the individual dyes providing these other components do not adversely affect the coloring result or have to be ruled out for other reasons, for example toxicological reasons.

So far as the dyes suitable for use in the hair colorants and tinting preparations according to the invention are concerned, reference is expressly made to the work by Ch. Zviak, The Science of Hair Care, Chapter 7 (pages 248-250; substantive dyes) and Chapter 8, pages 264-267; oxidation dye precursors), published as Volume 7 of the Series “Dermatology” (Ed.: Ch. Culnan and H. Maibach), Marcel Dekker Inc., New York/Basel, 1986, and to the “Europäische Inventar der Kosmetik-Rohstoffe” published by the Europäische Gemeinschaft and available on floppy disk from the Bundesverband Deutscher Industrie- und Handelsunternehmen für Arzneimittel, Reformwaren und Körperpflegemittel d.V., Mannheim.

Oxidation may be carried out with enzymes. In this case, the enzymes (enzyme class 1: oxidoreductases) can transfer electrons from suitable primary intermediates (reducing agents) to atmospheric oxygen. Oxidases, such as tyrosinase, ascorbate oxidase and laccase, are preferred for this purpose, as are glucoseoxidase, uricase or pyruvate oxidase. The enzymes may also be used to enhance the effect of an oxidizing agent present in small quantities. One example of such an enzymatic process is the procedure whereby the effect of small quantities (for example 1% and less, based on the formulation as a whole) of hydrogen peroxide is enhanced by peroxidases.

The colorants according to the invention may also contain any of the known active substances, additives and auxiliaries typical of such formulations. In many cases, the colorants contain at least one surfactant, both anionic and zwitterionic, ampholytic, nonionic and cationic surfactants being suitable in principle. In many cases, however, it has been found to be of advantage to select the surfactants from anionic, zwitterionic or nonionic surfactants.

Suitable anionic surfactants for the preparations according to the invention are any anionic surface-active substances suitable for use on the human body. Such substances are characterized by a water-solubilizing anionic group such as, for example, a carboxylate, sulfate, sulfonate or phosphate group and a lipophilic alkyl group containing around 10 to 22 carbon atoms. In addition, glycol or polyglycol ether groups, ester, ether and amide groups and hydroxyl groups may also be present in the molecule. The following are examples of suitable anionic surfactants—in the form of the sodium, potassium and ammonium salts and the mono-, di- and trialkanolammonium salts containing 2 or 3 carbon atoms in the alkanol group:

linear fatty acids containing 10 to 22 carbon atoms (soaps),

ether carboxylic acids corresponding to the formula R—O—(CH₂—CH₂O)ₓ—CH₂—COOH, in which R is a linear alkyl group containing 10 to 22 carbon atoms and x=0 or 1 to 16,

acyl sarcosides containing 10 to 18 carbon atoms in the acyl group,

acyl taurides containing 10 to 18 carbon atoms in the acyl group,

acyl isethionates containing 10 to 18 carbon atoms in the acyl group,

sulfosuccinic acid mono- and dialkyl esters containing 8 to 18 carbon atoms in the alkyl group and sulfosuccinic acid monoalkyl polyoxyethyl esters containing 8 to 18 carbon atoms in the alkyl group and 1 to 6 oxyethyl groups,

linear alkane sulfonates containing 12 to 18 carbon atoms,

linear α-olefin sulfonates containing 12 to 18 carbon atoms,

α-sulfofatty acid methyl esters of fatty acids containing 12 to 18 carbon atoms,

alkyl sulfates and alkyl polyglycol ether sulfates corresponding to the formula R—O(CH₂—CH₂O)ₓ—SO₃H, in which R is a preferably linear alkyl group containing 10 to 18 carbon atoms and x=0 or 1 to 12,

mixtures of surface-active hydroxysulfonates according to DE-A-37 25 030,

sulfated hydroxyalkyl polyethylene and/or hydroxyalkylene propylene glycol ethers according to DE-A-37 23 354,

sulfonates of unsaturated fatty acids containing 12 to 24 carbon atoms and 1 to 6 double bonds according to DE-A-39 26 344,

esters of tartaric acid and citric acid with alcohols in the form of addition products of around 2 to 15 molecules of ethylene oxide and/or propylene oxide with fatty alcohols containing 8 to 22 carbon atoms.

Preferred anionic surfactants are alkyl sulfates, alkyl polyglycol ether sulfates and ether carboxylic acids containing 10 to 18 carbon atoms in the alkyl group and up to 12 glycol ether groups in the molecule and, in particular, salts of saturated and, more particularly, unsaturated C₈₋₂₂ carboxylic acids, such as oleic acid, stearic acid, isostearic acid and palmitic acid.

Nonionic surfactants contain, for example, a polyol group, a polyalkylene glycol ether group or a combination of polyol and polyglycol ether groups as the hydrophilic group. Examples of such compounds are

products of the addition of 2 to 30 moles of ethylene oxide and/or 0 to 5 moles of propylene oxide onto linear fatty alcohols containing 8 to 22 carbon atoms, onto fatty acids containing 12 to 22 carbon atoms and onto alkylphenols containing 8 to 15 carbon atoms in the alkyl group,

C₁₂₋₂₂ fatty acid monoesters and diesters of products of the addition of 1 to 30 moles of ethylene oxide onto glycerol,

C₈₋₂₂ alkyl mono- and oligoglycosides and ethoxylated analogs thereof and

products of the addition of 5 to 60 moles of ethylene oxide onto castor oil and hydrogenated castor oil.

Preferred nonionic surfactants are alkyl polyglycosides corresponding to the general formula R¹O-(Z)ₓ. These compounds are characterized by the following parameters.

The alkyl group R¹ contains 6 to 22 carbon atoms and may be both linear and branched. Primary linear and 2-methyl-branched aliphatic groups are preferred. Such alkyl groups are, for example, 1-octyl, 1-decyl, 1-lauryl, 1-myristyl, 1-cetyl and 1-stearyl. 1-Octyl, 1-decyl, 1-lauryl and 1-myristyl are particularly preferred. Where so-called “oxo alcohols” are used as starting materials, compounds with an odd number of carbon atoms in the alkyl chain predominate.

The alkyl polyglycosides suitable for use in accordance with the invention may, for example, contain only one particular alkyl group R¹. However, such compounds are normally prepared from natural fats and oils or mineral oils. In this case, mixtures corresponding to the starting compounds or corresponding to the particular working up of these compounds are present as the alkyl groups R.

Particularly preferred alkyl polyglycosides are those in which R¹ consists

essentially of C₈ and C₁₀ alkyl groups,

essentially of C₁₂ and C₁₄ alkyl groups,

essentially of C₈ to C₁₆ alkyl groups or

essentially of C₁₂ to C₁₆ alkyl groups.

Any mono- or oligosaccharides may be used as the sugar unit Z. Sugars containing 5 or 6 carbon atoms and the corresponding oligosaccharides are normally used. Examples of such sugars are glucose, fructose, galactose, arabinose, ribose, xylose, lyxose, allose, altrose, mannose, gulose, idose, talose and sucrose. Preferred sugar units are glucose, fructose, galactose, arabinose and sucrose; glucose is particularly preferred.

The alkyl polyglycosides suitable for use in accordance with the invention contain on average 1.1 to 5 sugar units. Alkyl polyglycosides with x values of 1.1 to 1.6 are preferred. Alkyl glycosides where x is 1.1 to 1.4 are most particularly preferred.

Besides acting as surfactants, the alkyl glycosides may also be used to improve the fixing of perfume components to the hair. Accordingly, in cases where the effect of the perfume oil on the hair is intended to last longer than the duration of the hair treatment, alkyl glycosides will preferably be used as another ingredient of the preparations according to the invention.

Alkoxylated homologs of the alkyl polyglycosides mentioned may also be used in accordance with the invention. These homologs may contain on average up to 10 ethylene oxide and/or propylene oxide units per alkyl glycoside unit.

Zwitterionic surfactants may also be used, particularly as co-surfactants. In the context of the invention, zwitterionic surfactants are surface-active compounds which contain at least one quaternary ammonium group and at least one —COO⁽⁻⁾ or —SO₃⁽⁻⁾ group in the molecule. Particularly suitable zwitterionic surfactants are the so-called betaines, such as N-alkyl-N,N-dimethyl ammonium glycinates, for example cocoalkyl dimethyl ammonium glycinate, N-acylaminopropyl-N,N-dimethyl ammonium glycinates, for example cocoacylaminopropyl dimethyl ammonium glycinate, and 2-alkyl-3-carboxymethyl-3-hydroxyethyl imidazolines containing 8 to 18 carbon atoms in the alkyl or acyl group and cocoacylaminoethyl hydroxyethyl carboxymethyl glycinate. A preferred zwitterionic surfactant is the fatty acid amide derivative known by the CTFA name of Cocamidopropyl Betaine.

Also suitable, particularly as co-surfactants. are ampholytic surfactants. Ampholytic surfactants are surface-active compounds which, in addition to a C₈₋₁₈ alkyl or acyl group, contain at least one free amino group and at least one —COOH or —SO₃H group in the molecule and which are capable of forming inner salts. Examples of suitable ampholytic surfactants are N-alkyl glycines, N-alkyl propionic acids, N-alkyl aminobutyric acids, N-alkyl iminodipropionic acids, N-hydroxyethyl-N-alkyl amidopropyl glycines, N-alkyl taurines, N-alkyl sarcosines, 2-alkyl aminopropionic acids and alkyl aminoacetic acids containing around 8 to 18 carbon atoms in the alkyl group. Particularly preferred ampholytic surfactants are N-cocoalkyl aminopropionate, cocoacyl aminoethyl aminopropionate and C₁₂₋₁₈ acyl sarcosine.

According to the invention, the cationic surfactants used are particularly those of the quaternary ammonium compound, esterquat and amidoamine type. Preferred quaternary ammonium compounds are ammonium halides, more particularly chlorides and bromides, such as alkyl trimethyl ammonium chlorides, dialkyl dimethyl ammonium chlorides and trialkyl methyl ammonium chlorides, for example cetyl trimethyl ammonium chloride, stearyl trimethyl ammonium chloride, distearyl dimethyl ammonium chloride, lauryl dimethyl ammonium chloride, lauryl dimethyl benzyl ammonium chloride and tricetyl methyl ammonium chloride and the imidazolium compounds known under the INCI names of Quaternium-27 and Quaternium-83. The long alkyl chains of the above-mentioned surfactants preferably contain 10 to 18 carbon atoms.

Esterquats are known substances which contain both at least one ester function and at least one quaternary ammonium group as structural element. Preferred esterquats are quaternized ester salts of fatty acids with triethanolamine, quaternized ester salts of fatty acids with diethanol alkylamines and quaternized ester salts of fatty acids with 1,2-dihydroxypropyl dialkylamines. Such products are marketed, for example, under the names of Stepantex®, Dehyquart® and Armocare®. The products Armocare® VGH-70, an N,N-bis-(2-palmitoyloxyethyl)-dimethyl ammonium chloride, and Dehyquart® F-75 and Dehyquart® AU-35 are examples of such esterquats.

The alkyl amidoamines are normally prepared by amidation of natural or synthetic fatty acids and fatty acid cuts with dialkyl aminoamines. A compound from this group particularly suitable for the purposes of the invention is the stearamidopropyl dimethylamine obtainable under the name of Tegoamid® S 18.

Other cationic surfactants suitable for use in accordance with the invention are the quaternized protein hydrolyzates.

Also suitable for the purposes of the invention are cationic silicone oils such as, for example, the commercially available products Q2-7224 (manufacturer: Dow Corning; a stabilized trimethyl silyl amodimethicone), Dow Corning 929 Emulsion (containing a hydroxylamino-modified silicone which is also known as amodimethicone), SM-2059 (manufacturer: General Electric), SLM-55067 (manufacturer: Wacker) and Abil®-Quat 3270 and 3272 (manufacturer: Th. Goldschmidt; diquaternary polydimethyl siloxanes, Quaternium-80).

One example of a quaternary sugar derivative suitable for use as a cationic surfactant is the commercially available product Glucquat®100 (INCI name: Lauryl Methyl Gluceth-10 Hydroxypropyl Dimonium Chloride).

The compounds containing alkyl groups used as surfactants may be single compounds. In general, however, these compounds are produced from native vegetable or animal raw materials so that mixtures with different alkyl chain lengths dependent upon the particular raw material are obtained.

The surfactants representing addition products of ethylene and/or propylene oxide with fatty alcohols or derivatives of these addition products may be both products with a “normal” homolog distribution and products with a narrow homolog distribution. Products with a “normal” homolog distribution are mixtures of homologs which are obtained in the reaction of fatty alcohol and alkylene oxide using alkali metals, alkali metal hydroxides or alkali metal alcoholates as catalysts. By contrast, narrow homolog distributions are obtained when, for example, hydrotalcites, alkaline earth metal salts of ether carboxylic acids, alkaline earth metal oxides, hydroxides or alcoholates are used as catalysts. The use of products with a narrow homolog distribution can be of advantage.

The colorants according to the invention preferably may also contain a conditioning agent selected from the group consisting of cationic surfactants, cationic polymers, alkyl amidoamines, paraffin oils, vegetable oils and synthetic oils.

Cationic polymers can be preferred conditioning agents. These are generally polymers containing a quaternary nitrogen atom, for example in the form of an ammonium group. The following are examples of preferred cationic polymers:

Quaternized cellulose derivatives commercially available under the names of Celquat® and Polymer JR®. The compounds Celquat® H 100, Celquat® L 200 and Polymer JR®400 are preferred quaternized cellulose derivatives.

Polymeric dimethyl diallyl ammonium salts and copolymers thereof with acrylic acid and with esters and amides of acrylic acid and methacrylic acid. The products commercially available under the names of Merquat®100 (poly(dimethyl diallyl ammonium chloride)), Merquat®550 (dimethyl diallyl ammonium chloride/acrylamide copolymer) and Merquat® 280 (dimethyl diallyl ammonium chloride/acrylic acid copolymer) are examples of such cationic polymers.

Copolymers of vinyl pyrrolidone with quaternized derivatives of dialkylaminoacrylate and methacrylate, such as vinyl pyrrolidone/dimethylaminomethacrylate copolymers quaternized, for example, with diethyl sulfate. Compounds such as these are commercially available under the names of Gafquat®734 and Gafquat®755.

Copolymers of vinyl pyrrolidone with methoimidazolinium chloride which are commercially available under the name of Luviquat®.

Quaternized polyvinyl alcohol.

The polymers with quaternary nitrogen atoms in the main polymer chain known by the names of Polyquaternium 2, Polyquaternium 17, Polyquaternium18 and Polyquaternium 27.

Cationic polymers from the first four groups mentioned are particularly preferred, Polyquaternium 2, Polyquaternium 10 and Polyquaternium 22 being most particularly preferred.

In a particularly preferred embodiment, zwitterionic or ampholytic polymers are used alternatively to the cationic polymers as conditioning agents. Preferred representatives are octylacrylamide/methyl methacrylate/tert.butyl aminoethyl methacrylate/2-hydroxypropyl methacrylate copolymers and in particular the acrylamidopropyl trimethyl ammonium chloride/acrylate copolymer.

Other suitable conditioning agents are silicone oils, more particularly dialkyl and alkylaryl siloxanes, such as for example dimethyl polysiloxane and methylphenyl polysiloxane, and alkoxylated and quaternized analogs thereof. Examples of such silicones are the products marketed by Dow Corning under the names of DC 190, DC 200, DC 344, DC 345 and DC 1401 and the products Q2-7224 (manufacturer: Dow Corning; a stabilized trimethyl silyl amodimethicone), Dow Corning® 929 Emulsion (containing a hydroxylamino-modified silicone which is also known as amodimethicone), SM-2059 (manufacturer: General Electric), SLM-55067 (manufacturer: Wacker) and Abil® Quat 3270 and 3272 (manufacturer: Th. Goldschmidt; diquaternary polydimethyl siloxanes, Quaternium-80).

Other suitable conditioning agents are paraffin oils, synthetically produced oligomeric alkenes and vegetable oils, such as jojoba oil, sunflower oil, orange oil, almond oil, wheatgerm oil and peach kernel oil.

Phospholipids, for example soya lecithin, egg lecithin and kephalins, are also suitable hair-conditioning compounds.

Other active substances, auxiliaries and additives are, for example,

nonionic polymers such as, for example, vinyl pyrrolidone/vinyl acrylate copolymers, polyvinyl pyrrolidone and vinyl pyrrolidone/vinyl acetate copolymers and polysiloxanes,

anionic polymers such as, for example, polyacrylic acids, crosslinked polyacrylic acids, vinyl acetate/crotonic acid copolymers, vinyl pyrrolidone/vinyl acrylate copolymers, vinyl acetate/butyl maleate/isobornyl acrylate copolymers, methyl vinyl ether/maleic anhydride copolymers and acrylic acid/ethyl acrylate/N-tert.butyl acrylamide terpolymers,

thickeners, such as agar agar, guar gum, alginates, xanthan gum, gum arabic, karaya gum, locust bean gum, linseed gums, dextrans, cellulose derivatives, for example methyl cellulose, hydroxyalkyl cellulose and carboxymethyl cellulose, starch fractions and derivatives, such as amylose, amylopectin and dextrins, clays such as, for example, bentonite or fully synthetic hydrocolloids such as, for example, polyvinyl alcohol,

structurants, such as maleic acid and lactic acid,

hair-conditioning compounds, such as phospholipids, for example soya lecithin, egg lecithin and kephalins,

protein hydrolyzates, more particularly elastin, collagen, keratin, milk protein, soya protein and wheat protein hydrolyzates, condensation products thereof with fatty acids and quaternized protein hydrolyzates,

perfume oils, dimethyl isosorbide and cyclodextrins,

solvents and solubilizers, such as ethanol, isopropanol, ethylene glycol, propylene glycol, glycerol and diethylene glycol,

fiber-restructuring agents, more particularly mono-, di- and oligosaccharides such as, for example, glucose, galactose, fructose and lactose,

quaternized amines, such as methyl-1-alkylamidoethyl-2-alkylimidazolinium methosulfate,

defoamers, such as silicones,

antidandruff agents, such as piroctone olamine, zinc omadine and climbazol,

UV filters, more particularly derivatized benzophenones, cinnamic acid derivatives and triazines,

substances for adjusting the pH value, for example typical acids, more particularly food-grade acids and bases,

active substances, such as allantoin, pyrrolidone carboxylic acids and salts thereof and bisabolol,

vitamins, provitamins and vitamin precursors, more particularly those of groups A, B₃, B₅, C, E, F and H,

plant extracts, such as the extracts of green tea, oak bark, stinging nettle, hamamelis, hops, chamomile, burdock root, horse willow, hawthorn, lime blossom, almond, aloe vera, pine needle, horse chestnut, sandalwood, juniper, coconut, mango, apricot, lemon, wheat, kiwi, melon, orange, grapefruit, sage, rosemary, birch, mallow, lady's smock, creeping thyme, yarrow, thyme, balm, restharrow, coltsfoot, hibiscus, meristem, ginseng and ginger root,

cholesterol,

consistency factors, such as sugar esters, polyol esters or polyol alkyl ethers,

fats and waxes, such as spermaceti, beeswax, montan wax and paraffins,

fatty acid alkanolamides,

complexing agents, such as EDTA, NTA, β-alanine diacetic acid and phosphonic acids,

swelling and penetration agents, such as glycerol, propylene glycol monoethyl ether, carbonates, hydrogen carbonates, guanidines, ureas and primary, secondary and tertiary phosphates,

opacifiers, such as latex, styrene/PVP and styrene/acrylamide copolymers,

pearlizers, such as ethylene glycol mono- and distearate and PEG-3-distearate,

pigments,

stabilizers for hydrogen peroxide and other oxidizing agents,

propellents, such as propane/butane mixtures, N₂O, dimethyl ether, CO₂ and air,

antioxidants.

Information on other optional components and the quantities in which they are used can be found in the reference books known to the expert, for example Kh. Schrader, Grundlagen und Rezepturen der Kosmetika, 2nd Edition, Hüthig Buch Verlag, Heidelberg, 1989.

A second embodiment of the present invention is a process for tinting and/or coloring keratin fibers, more particularly human hair, in which

if desired, a pretreatment preparation M1 is applied to the fibers,

a tint or colorant M2 is then used on the fibers, another preparation M3 optionally being added to M2 before application,

the colorant M2 is rinsed from the fibers after a contact time of 5 to 30 minutes and

after the treatment, an aftertreatment preparation M4 is optionally applied to the fibers and is rinsed from them after a contact time of a few minutes,

characterized in that at least one of the preparations M1, M2, M3 or M4 contains at least one compound corresponding to formula (I).

The preparation M1 used in this process is a pretreatment preparation containing at least one compound corresponding to formula (I) in a cosmetic carrier. The colorant M2 is a colorant according to any of claims 1 to 9. A preparation M3 containing at least one compound corresponding to formula (I) is preferably added to the colorant M2 shortly before application. The preparation M3 containing at least one compound corresponding to formula (I) may be made up both as a solid and as an aqueous solution.

According to the invention, the aftertreatment preparation M4 is a hair aftertreatment preparation as described, for example, in K. Schrader, Grundlagen und Rezepturen der Kosmetika, 2nd Edition, 1989, Dr. Alfred Hüthig Verlag, Heidelberg, pp. 722 et seq. which may optionally contain at least one compound corresponding to formula (I).

A third embodiment of the invention is the use of a preparation in a process for coloring or tinting keratin fibers, more particularly human hair, which contains at least one substantive dye and also at least one compound corresponding to formula (I):

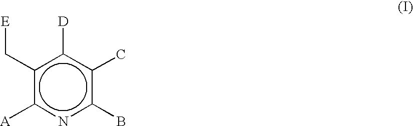

in which

A and B independently of one another represent hydrogen, halogen, a C₁₋₄ alkyl group, a C₃₋₆ cycloalkyl group, a C₁₋₄ monohydroxyalkyl group, a C₂₋₄ oligohydroxyalkyl group, a C₁₋₄ aminoalkyl group, a group —OR or a group —NR¹R², where R¹ and R² independently of one another represent hydrogen, a C₁₋₄ alkyl group or a C₁₋₄ monohydroxyalkyl group or R¹ and R² together with the nitrogen atom form a saturated ring,

C represents a group —OR, —NR¹R², —OP(O)(OR³)₂, a C₁₋₄ monohydroxyalkyl group, a C₂₋₄ oligohydroxyalkyl group or a C₁₋₄ alkyl group,

D represents a group —OR, a carboxy group, a C₁₋₂₂ alkoxycarbonyl group, a formyl group, a group —CH₂OR or a group —CH₂—NR₂,

E represents a group —OR, —OP(O)(OR³)₂, a C₁₋₄ monohydroxyalkyl group or a C₂₋₄ oligohydroxyalkyl group,

R representing hydrogen, a C₁₋₄ alkyl group, a C₁₋₂₂ acyl group, a hydroxy-C₂₋₂₂-acyl group, a C₂₋₁₀ carboxyacyl group, a C₃₋₁₀ oligocarboxyacyl group, an oligocarboxymonohydroxy-C₃₋₁₀-acyl group, an oligocarboxyoligohydroxy-C₃₋₁₀-acyl group, a C₃₋₈ cycloalkyl group, a C₁₋₄ monohydroxyalkyl group, a C₂₋₄ oligohydroxyalkyl group, an aryl group which may contain a hydroxy, nitro or amino group, a heteroaromatic group or a group —CH₂CH₂NR¹R², where R¹ and R² are as defined above,

R³ representing hydrogen or a C₁₋₅ alkyl group,

or one of the corresponding physiologically compatible salts.

The following Examples are intended to illustrate the invention.

### EXAMPLES

I) Coloring

Colorants were formulated using two different carriers.

| Colorant 1 | | |
|---|---|---|
| Mixture A | | |
| Hydrenol ® D¹ | 3.00 | g |
| Kokoslorol ® C12-18² | 2.50 | g |
| Akypo ® RLM 45N³ | 3.00 | g |
| Propyl p-hydroxybenzoate | 0.15 | g |
| Methyl p-hydroxybenzoate | 0.15 | g |
| Phenoxyethanol | 0.50 | g |
| Carbopol ® ETD 2001⁴ | 0.20 | g |
| Vitamin E acetate | 0.20 | g |
| AMP 95⁵ | to pH 7.0 | |
| Water | 70.00 | g |

¹C₁₆₋₁₈ fatty alcohol (INCI name: Cetearyl Alcohol) (COGNIS)
²C₁₂₋₁₈ fatty alcohol (INCI name: Coconut Alcohol) (COGNIS)
³Lauryl alcohol + ca. 4.5 mol ethylene oxide/acetic acid sodium salt (ca. 82% active substance (INCI name: Sodium Laureth-6 Carboxylate)) (KAO, Chem-Y)
⁴Polyacrylic acid homopolymer (INCI name: Carbomer) (GOODRICH)
⁵2-Amino-2-methylpropanol (95% active substance, 5% water) (CSC CHEMIE)

The substances were melted at 80° C., mixed with water heated to 80° C. and emulsified with vigorous stirring. The emulsion was then cooled with gentle stirring. The remaining components were added with stirring at a temperature of 40° C. A pH of 7 was then adjusted with AMP.

| Mixture B | |
|---|---|
| Ammonium sulfate | 1.00 g |
| Substantive dyes; | |
| Table (I) (Examples D1-D5) | 1.00-3.00 g |
| Vitamin B6 (pyridoxine · HCl) | 1.00 g |
| Water | 10.00 g |

The dye was dissolved in water heated to 50° C. while ammonium sulfate, tartaric acid and vitamin B6 (pyridoxine HCl) were added. The dye solution (mixture B) was added to the emulsion (mixture A) and made up with water to 100 g. Stirring was then continued until the temperature reached room temperature.

| Colorant 2 | | |
|---|---|---|
| Mixture A | | |
| Hydrenol ® D¹ | 1.50 | g |
| Kokoslorol ® C12-18² | 3.50 | g |
| Texapon ® NSO F³ | 15.00 | g |
| Polychol ® 5⁴ | 0.40 | g |
| Luviskol ® K 30⁵ | 0.50 | g |
| Ammonia (25% aqueous solution) | to pH 9 | |
| Water | 55.00 | g |

¹C₁₆₋₁₈ fatty alcohol (INCI name: Cetearyl Alcohol) (COGNIS)
²C₁₂₋₁₈ fatty alcohol (INCI name: Coconut Alcohol) (COGNIS)
³Lauryl ether sulfate sodium salt (INCI: Sodium Laureth Sulfate) (COGNIS)
⁴Lanolin alcohol + 5EO (INCI name: LANETH-5) (CRODA)
⁵Polyvinyl pyrrolidone (INCI: PVP) (BASF)

The substances Hydrenol® D, Kokoslorol®C12-18, Texapon® NSO F and Polychol® 5 were melted at 80° C., mixed with water heated to 80° C. and emulsified with vigorous stirring. The AMPD and the Luviskol® K 30 were then added and the emulsion was cooled with gentle stirring.

| Mixture B | |
|---|---|
| Ammonium sulfate | 1.00 g |
| Substantive dyes; | |
| Table (I) (Examples D6-D10) | 1.00-3.00 g |
| Vitamin B6 (pyridoxine · HCl) | 1.00 g |
| Water | 10.00 g |

The dye was dissolved in water heated to 50° C. while ammonium sulfate and optionally a little ammonia were added. The dye solution (mixture B) was added to the emulsion (mixture A), adjusted with ammonia to pH 9 and made up with water to 100 g. Stirring was then continued until the temperature reached room temperature.

TABLE 1

| | D1 | D2 | D3 | D4 | D5 | D6 | D7 | D8 | D9 | D10 |
|---|---|---|---|---|---|---|---|---|---|---|
| F1 | 0.05 | | | | | | | | | |
| F2 | | | | 0.05 | | | | | | |
| F3 | | | | | | 0.30 | | | | 0.10 |
| F4 | | | | | | | | 0.10 | | |
| F5 | 0.40 | | | 0.02 | 0.20 | 1.00 | 0.60 | | | 1.50 |
| F6 | 0.20 | | 0.50 | 0.50 | | | | | 0.30 | 0.30 |
| F7 | 0.60 | | 0.50 | | 0.01 | | 0.10 | | 0.30 | |
| F8 | | 0.60 | 0.20 | 0.05 | 0.05 | 1.00 | 0.20 | 0.05 | | 0.60 |
| F9 | | | | 0.08 | | | | | 0.15 | 0.60 |
| F10 | | 0.80 | 0.20 | 0.02 | 0.25 | | 0.30 | 0.50 | 0.60 | |
| Colorant | 1 | 1 | 1 | 1 | 1 | 2 | 2 | 2 | 2 | 2 |

F1 Arianor Mahogany
F2 Arianor Sienna Brown
F3 Anianor Steel Blue
F4 Fluor yellow
F5 HC Blue 2
F6 HC Red 3
F7 HC Red BN
F8 HC Violet
F9 HC Yellow 2
F10 Rodol 9R Base

Coloring

The colorants obtained were applied to 5 cm long tresses of standardized, 80% gray, but not specially pretreated human hair and left thereon for 30 mins. at 32° C. The hair was then rinsed, washed with a standard shampoo and then dried.

The results of the coloring tests are set out in Table 2.

TABLE 2

| Formulation (see Table 1) | Hair color |
|---|---|
| D1 | Garnet brown |
| D2 | Ruby red |
| D3 | Violet red |
| D4 | Wheat |
| D5 | Hazelnut |
| D6 | Dark violet |
| D7 | Chocolate |
| D8 | Loam-colored |
| D9 | Copper |
| D10 | Brown |

II). Demonstration of the Structuring Effect of Vitamin B6 When Applied Together with the Colorant

A) Analysis Method Used: HP-DSC (High-Pressure Differential Scanning Calorimetry)

Thermoanalytical investigations are particularly suitable for characterizing two-phase systems to which human hair fibers as fibrous keratins with their crystalline α-helix component and amorphous matrix component also belong. On the one hand, glass transitions and aging behavior of the amorphous matrix can be investigated, on the other hand the melting behavior of the crystalline helical phase provides important information. Thermoanalytical studies were described for the first time in 1899. The first differential thermoanalyses (DTA) of protein fibers were carried out towards the end of the fifties (F. Schwenker, J. H. Dusenbury, Text. Res. J. 1963, 30, pages 800 et seq; W. D. Felix, M. A. McDowall, H. Eyring, ibid. (1963), 33, pages 465 et seq). In the following years, various thermoanalytical measuring techniques, such as DTA, HP-DTA (high-pressure DTA) and DSC (differential scanning calorimetry) were applied to keratin fibers, for example to investigate the phenomenon of supercontraction, α-β-phase transitions of the helices or denaturing processes. Recently, the method of IIP-DSC was used to study keratin fibers, more particularly at the Deutsches Wollforschungsinstitut (German Wool Research Institute) in Aachen (F. J. Wortmann, H. Deutz, J. Appl. Polym. Sci. 1993, 48, pp. 137 et seq.) IIP-DSC rules out the problems associated with pyrolytic effects which occur in conventional DSC and the problems with data acquisition and interpretation by which DTA is attended. DSC measurements are carried out on keratins which are encapsulated with water in commercially obtainable pressure-tight measuring capsules. In the keratin/water system, a high water vapor pressure from which the HP-DSC analysis derives builds up in the encapsulated steel crucibles on heating to >100° C. The crucial difference between the HP-DSC thermograms of human hair fibers and normal DSC thermograms is that the endothermal peaks which reproduce the transition point and the transition enthalpy are shifted by ca. 90° C. to lower temperatures. This derives from the fact that, after diffusing into the hair fibers, the water reduces protein stability by weakening and splitting hydrogen bridge bonds and salt bonds so that the “gluing temperature” of the keratins is reduced. If only hydrogen bridges and salt bridges are dissolved by the supercontracting agent, such as water, the thermal effect is reversible (supercontraction). However, the process becomes irreversible when covalent bonds, such as disulfide bridges for example, are split. This happens when human hair fibers are heated with water to >150° C. in pressure-tight capsules. The irreversible transition, interpreted as the transition of the α-helical regions in the proteins into a random state, results in endothermal peaks, the position of the peaks reproducing the transition point or even the denaturing point and their area reproducing the transition or denaturing enthalpy.

Accordingly, both structural and chemical states and changes in fiber keratins and particularly in human hair fibers can be detected by dynamic differential scanning calorimetry (DSC). Under precisely defined test conditions, the processes detectable by calorimetry in human hair fibers can be recorded in the form of thermograms and used in regard to peak positions, structures and areas as an indicator for influencing order/disorder transitions through changes in inner and/or outer parameters produced, for example, by cosmetic treatment of the hair fibers. In other words, information on the strength of or damage to human hair fibers can be obtained from the endothermal peaks recorded in the thermogram of human hair fibers on the basis of peak position (transition point) and peak area (transition enthalpy).

Detailed investigations into the influence of the cystine content on the denaturing of the α-helices in keratins have, for example, shown that the denaturing temperature (transition temperature) of the keratin increases linearly with the cystine content. The effect of the increased stability of the matrix region through the higher degree of crosslinking of the increased percentage of disulfide bridges in the matrix is that the transition of the helices embedded in this matrix is made difficult, resulting in an increase in the denaturing temperature. Conversely, a reduction in the denaturing temperature and above all in the denaturing enthalpy can generally be observed in human hair fibers treated by permanent waving or bleaching or coloring (H. Deutz, Doktorarbeit, RWTH Aachen 1993).

B) Procedure

Human hair (Alkinco 6634) was intentionally damaged by permanent waving (commercial product Poly Lock extra starke Dauerwelle; 40 mins. permanent waving, 10 mins. fixing). The pretreated hair was then colored with a colorant with different contents of pyridoxine.HCl. The denaturing temperatures of the colored hair samples was thermoanalytically determined by HP-DSC.

Quantities of 4.0 g of formulations D2 and D8 prepared in accordance with 1) (containing 1% pyridoxine and without pyridoxine) were applied to 0.5 g of damaged human hair. After a contact time of 30 minutes at 32° C., the hair was rinsed and then dried. The active ingredients were then tested for restructuring effects by HP-DSC measurements. The denaturing temperatures obtained are listed in Table 3. Table 3.

| Example | Formulation | Denaturing temperature [° C.] |
|---|---|---|
| II. 1 | None (untreated hair) | 145.8 |
| II. 2 | D2 without pyridoxine · HCl | 146.0 |
| II. 3 | D2 | 148.3 |
| II. 4 | D8 without pyridoxine · HCl | 146.2 |
| II. 5 | D8 | 148.7 |

What is claimed:

1. A preparation containing at least one substantive dye for coloring keratin fibers which additionally contains at least one compound of the formula: 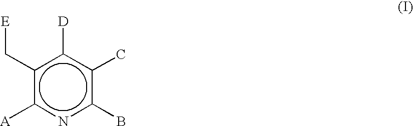
wherein
A and B independently of one another are selected from the group consisting of hydrogen, halogen, a C₁₋₄ alkyl group, a C₃₋₆ cycloalkyl group, a C₁₋₄ monohydroxyalkyl group, a C₂₋₄ oligohydroxyalkyl group, a C₁₋₄ aminoalkyl group, a group NR¹R² and a group —OR wherein R is selected from the group consisting of hydrogen, a C₁₋₄ alkyl group, a C₁₋₂₂ acyl group, a hydroxyl-C₂₋₂₂-acyl group. a C₂₋₁₀ carboxyacyl group, a C₃₋₁₀ oligocarboxyacyl group, an oligocarboxymonohydroxy-C₃₋₁₀-acyl group, an oligocarboxyoligohydroxy-C₃₋₁₀-acyl group, a C₃₋₈ cycloalkyl group, a C₁₋₄ monohydroxyalkyl group, a C₂₋₄ oligohydroxyalkyl group, an aryl group which may contain a hydroxyl, nitro or amino group, a heteroaromatic group or a group —CH₂CH₂NR¹R² where R¹ and R² are selected from the group consisting of hydrogen, a C₁₋₄ alkyl group and a C₁₋₄ monohydroxyalkyl group or R¹ and R² together with the nitrogen atom form a saturated ring;
C is selected from the group consisting of —OR, —NR¹R², —OP(O)(OR³)₂, a C₁₋₄ monohydroxyalkyl group, a C₂₋₄ oligohydroxyalkyl group and a C₁₋₄ alkyl group wherein R¹ and R² are defined above and R³ is hydrogen or a C₁₋₅ alkyl group;
D is selected from the group consisting of —OR, a carboxy group, a C₁₋₂₂ alkoxycarbonyl group, a formyl group, a group —CH₂OR and a group —CH₂—NR² wherein R and R² are defined above;
E is selected from the group consisting of —OR, —OP(O)(OR³)₂, a C₁₋₄ monohydroxyalkyl group and a C₂₋₄ oligohydroxyalkyl group wherein R and R³ are defined above,
or one of the corresponding physiologically compatible salts thereof.

2. The preparation of claim 1, wherein one of the two groups A and B is hydrogen.

3. The preparation of claim 2, wherein one of the two groups A and B is hydrogen and the other is a C₁₋₄ alkyl group.

4. The preparation of claim 3, wherein C is selected from the group consisting of a hydroxy group, a C₁₋₄ monohydroxyalkyl group and a C₂₋₄ oligohydroxyalkyl group.

5. The preparation of claim 4, wherein D is selected from the group consisting of a hydroxymethyl group, a hydroxy group, a carboxy group, a group —CH₂—NR₂ and a formyl group.

6. The preparation of claim 5, wherein E is selected from the group consisting of a hydroxy group, a C₁₋₄ monohydroxyalkyl group and a group —OP(O)(OH)₂.

7. The preparation of claim 6, wherein the compound corresponding to formula (I) is selected from the group consisting of pyridoxine, pyridoxal, pyridoxal-5′-phosphate and pyridoxamine.

8. The preparation of claim 7, wherein the substantive dye is selected from the group consisting of HC Yellow 2, HC Yellow 4, HC Yellow 5, HC Yellow 6, HC Yellow 12, HC Orange 1, Disperse Orange 3, HC Red 1, HC Red 3, HC Red 10, HC Red 11, HC Red 13, HC Red BN, HC Blue 2, HC Blue 12, Disperse Blue 3, HC Violet 1, Disperse Violet 1, Disperse Violet 4, Acid Violet 43, Disperse Black 9 and Acid Black 52 and also 1,4-diamino-2-nitrobenzene, 2-amino-4-nitrophenol, 1,4-bis-(β-hydroxyethyl)-amino-2-nitrobenzene, 3-nitro-4-(β-hydroxyethyl)-aminophenol, 2-(2′-hydroxyethyl)-amino-4,6-dinitrophenol, 1-(2′-hydroxyethyl)-amino-4-methyl-2-nitro-benzene, 1-amino-4-(2′-hydroxyethyl)-amino-5-chloro-2-nitrobenzene, 4-amino-3-nitrophenol, 1-(2′-ureidoethyl)-amino-4-nitrobenzene, 4-amino-2-nitrodiphenylamine-2′-carboxylic acid, 6-nitro-1,2,3,4-tetrahydroquinoxaline, 2-hydroxy-1,4-naphthoquinone, picramic acid and salts thereof, 2-amino-6-chloro-4-nitrophenol, 4-ethylamino-3-nitrobenzoic acid and 2-chloro-6-ethylamino-1-hydroxy-4-nitrobenzene.

9. The preparation of claim 8, wherein it additionally contains at least one oxidation dye precursor of the primary intermediate type and optionally at least one oxidation dye precursor of the secondary intermediate type.

10. A process for tinting and/or coloring keratin fibers comprising
(a) if desired, applying a pretreatment preparation M1 to the fibers,
(b) applying a tint or colorant M2 on the fibers, another preparation M3 optionally being added to M2 before application,
(c) rinsing the colorant M2 from the fibers after a contact time of 5 to 30 minutes and
(d) after the treatment, optionally applying an aftertreatment preparation M4 to the fibers and is rinsed from them after a contact time of a few minutes,
wherein at least one of the preparations M1, M2, M3 or M4 contains at least one compound corresponding to formula (I).

11. The process of claim 10, wherein a preparation M3 containing at least one compound corresponding to formula (I) is added to the colorant M2 shortly before application.

12. The process of claim 11, wherein the colorant M2 is a preparation of claim 1.

13. The use of the preparation of claim 1 in a process for tinting and/or coloring keratin fibers.
